(19) United States
(12) Patent Application Publication
(10) Pub. No.: US 2012/0283499 A1
(43) Pub. Date: Nov. 8, 2012

(54) METHOD FOR PRODUCING DISTILLATES BY MEANS OF CATALYTIC OLIGOMERIZATION OF OLEFINS IN THE PRESENCE OF OXYGENATED COMPOUNDS

(75) Inventors: Nikolai Nesterenko, Nivelles (BE); Delphine Minoux, Nivelles (BE); Sander Van Donk, Sainte-Adresse (FR); Jean-Pierre Dath, Beloeil Hainaut (BE)

(73) Assignee: TOTAL RAFFINAGE MARKETING, Puteaux (FR)

(21) Appl. No.: 13/501,693

(22) PCT Filed: Oct. 13, 2010
(86) PCT No.: PCT/FR2010/052167

§ 371 Date: Jul. 6, 2012

(30) Foreign Application Priority Data
Oct. 13, 2009 (FR) 09 57152

(51) Int. Cl. C07C 2/06

(52) U.S. Cl. 585/520

(57) ABSTRACT

The invention relates to a method for producing distillates by means of oligomerization using a C2C10 hydrocarbon filler and at least one organic oxygenated compound containing at least one oxygen atom and at least two carbon atoms. By including the addition of a substantial amount of oxygenated compounds, said method enables a reduction in the amount of olefins having chain lengths that are too short to allow the use thereof (typically in C10, or even less) and an increase in the yields of molecules in C10+, with controlled exothermicity of the oligomerization reactions.

The invention relates to a process for producing distillates by oligomerization starting with a C2-C10 hydrocarbon-based charge and at least one oxygenated organic compound containing at least one oxygen atom and at least two carbon atoms.

The term “distillate” means hydrocarbons containing 10 or more carbon atoms, middle distillates comprising from 10 to 20 carbon atoms and distilling in the temperature range from 145° C. to 350° C. Among the distillates, C10-C12 olefins (jet fuel) and C12+ olefins (diesel) will especially be distinguished.

Processes for the catalytic oligomerization of olefins are addition processes of olefin molecules for increasing the number of carbon atoms (or chain length) of the olefins.

The majority of the processes described in the bibliography propose solutions for oligomerizing a charge rich in olefins and more particularly in C3-C4 olefins. These olefins may be converted into oligomers under relatively mild conditions in which the contribution of the cracking reaction is negligible. The effect of the presence in the charge of a broader range of olefins in combination with an amount of inert or semi-inert material (paraffins, naphthenes and aromatics) makes the application of standard oligomerization processes less efficient.

Due to the different reactivity of C2-C10 olefins and above all due to the lower reactivity of C5-C10 olefins, it may be necessary to work at a higher temperature in order to improve the direct yield in an oligomerization process. Working at a higher temperature may lead to the cracking and aromatization of already-formed oligomers from more reactive olefins as long as part of the olefins of the charge still remains unconverted. In this case, the products of the C2-C10 olefin oligomerization processes contain large amounts of olefins bearing an excessively short chain length (less than 10 carbon atoms). These olefins cannot be used directly and must be recycled into the process in order to increase their chain length. This recycling, which may be up to 75% of the effluent produced, increases the complexity and costs of the installation. In addition, the recycling requires an additional step in the separation of olefins with a short chain length of the corresponding paraffins. The fact that these molecules have similar IPBs (initial distillation points) does not make it possible to use distillation, and necessitates the use of more sophisticated processes to separate them.

The present invention provides a solution for improving the process for the oligomerization of olefinic charge containing a large amount of inert material (paraffins, naphthenes and aromatics) and olefins with different reactivity.

The presence of oxygenated molecules as water precursors leads to moderation of the acidity of the oligomerization catalyst, limiting the cracking reaction, and gives a solution for managing the exothermicity of the oligomerization reaction.

On account of a lower reactivity of heavy olefins relative to light olefins in oligomerization, the presence of an oxygenated molecule as precursor for a light olefin generated progressively may lead to a higher conversion of the heavy olefins, resulting in a product whose chain length is longer.

There is thus a need to reduce, or even eliminate, these recycling operations, by increasing the yield of distillates, especially of C10-C20 middle distillates.

Moreover, oligomerization reactions are highly exothermic, which requires a control of the temperature of oligomerization units. This control may be performed with a unit using a system of several reactors, with cooling devices between reactors.

The invention is directed toward overcoming these drawbacks by proposing a process for the catalytic oligomerization of C2-C10 olefins, which makes it possible, by addition of oxygenated compounds, to reduce the amounts of olefins with a two short chain length to be exploited (typically C10, or even less) and to increase the yields of C10+ molecules, while at the same time controlling the exothermicity of the oligomerization reactions.

The invention thus allows an appreciable reduction in recycling operations, or even suppression thereof.

Document U.S. Pat. No. 7,183,450 describes a process for oligomerizing a charge comprising C2-C12 olefins and oxygenated compounds, the concentration of the latter compounds in the charge being between 1000 ppm and 10% by weight. The charge contains at least 50% linear monoolefins, these linear monoolefins having a C6+ content not exceeding 20%. The oligomerization reaction is performed at a reaction temperature of from 250 to 325° C., at a pressure of from 50 bar to 500 bar, the harshest conditions being necessary when the content of oxygenated compounds is the highest.

Surprisingly, the Applicant has discovered that, for a hydrocarbon-based charge, the addition of an amount (greater than or equal to 0.5% by weight relative to the hydrocarbon-based charge) of one or more oxygenated compounds comprising at least two carbon atoms makes it possible to increase the selectivity of the catalytic oligomerization process toward C10+, the degree of oligomerization being higher relative to the same charge under similar conditions, in the absence of such oxygenated compounds.

To this end, a first subject of the invention relates to a process for producing distillates from a hydrocarbon-based charge containing C2-C10 olefins, in which the treatment of the charge comprises at least one step of oligomerization of the charge performed in at least one oligomerization reactor, in which the charge is oligomerized in the presence of at least 0.5% by weight of at least one oxygenated organic compound containing at least one oxygen atom and at least two carbon atoms, this oxygenated compound possibly being of plant origin.

Advantageously, this oxygenated organic compound is derived from a synthetic step performed before the oligomerization step.

Preferably, the charge contains not more than 70% by weight of organic compound(s), advantageously from 15% to 70% by weight, preferably from 0.5% to 50% by weight and more particularly from 1% to 30% by weight.

The oxygenated organic compound(s) will be chosen, for example, among alcohols, ethers (with the exception of dimethyl ether) and carbonyl compounds, especially of C2-C20 and preferably of C2-C8, and the corresponding ethers, these compounds being chosen alone or as mixtures.

Organic compounds that are suitable for use include, for example, without being limited thereto, ethanol, n-propanol, isopropanol, n-butanol, isobutanol; diethyl ether, methyl ethyl ether, diisopropyl ether, dimethyl carbonate, dimethyl ketone and acetic acid, and mixtures thereof.

The preferred compounds are chosen among alcohols and ethers, preferably among ethanol, propanol, isopropanol, butanol, isobutanol, glycerol, ethylene glycol and the corresponding ethers, alone or as mixtures.

Advantageously, before the oligomerization, a step of synthesis of the oxygenated compound(s) is performed, all the products obtained during this synthesis being optionally mixed with the charge for the oligomerization step, or the oxygenated molecules contained in the products obtained during this synthetic step being isolated before being mixed with the charge.

The oxygenated compound(s) are obtained, for example, by conversion of biomass. This makes it possible to incorporate compounds of biological origin into the oligomerization products.

The synthesis of oxygenated molecules from biomass may be performed via synthesis gas, a pyrolysis in the absence of oxygen, hydropyrolysis, a transetherification or an anaerobic or aerobic fermentation. The oxygenated molecules may be isolated or used as a mixture. The oxygenated molecules used may undergo a pretreatment in order to reduce their content of metal ions and of nitrogenous compounds.

Bioethanol is the source of biocarbon that is the most widely used at the present time. It is, however, currently difficult to prepare C10+ distillates, which in particular sparingly branched, from bioethanol. The known processes require high recycling rates, heavy investments in C2 extraction equipment and substantial purging: a large amount of the bioethanol is thus found in the form of a light fraction (LPG, liquefied petroleum gas) of low added value.

The process according to the invention thus makes it possible to incorporate bioethanol into the charge in order to obtain hydrocarbons that are rich in distillates containing biocarbon substantially free of oxygenated compounds.

The oxygenated compound(s) may be obtained by condensation, in a separate reaction zone, of light oxygenated molecules possibly originating from the biomass. These light oxygenated molecules are chosen from oxygenated compounds containing 1 or 2 carbon atoms, such as ethanol, or DEE, or mixtures thereof.

Such a reaction is performed, for example, by placing ethanol in contact in an aqueous phase with at least one basic catalyst, at a temperature and a pressure that are sufficient to obtain a liquid effluent containing at least 40% by weight and preferably at least 50% by weight of molecules with three or more carbon atoms, or less than 10% by weight of ethylene. The rest of the effluent comprises unconverted oxygenated molecules. These molecules are either recycled into the condensation reactor, or are supplied for the oligomerization with the remaining effluent. The ethanol may be subjected alone to the condensation, or in the presence of methanol, DME, DEE, formaldehyde, acetaldehyde or ethylene glycol (oxygenated molecules containing one or two carbon atoms) or mixtures thereof.

The weight ratio of the effluent derived from the condensation of light oxygenated compounds (for example ethanol) and of the charge containing C2-C10 olefins will be, for example, from 0.005 to 1000 and preferably from 0.01 to 100.

The various oxygenated compounds described above may be used alone or as mixtures.

The hydrocarbon-based charge used may be a mixture of hydrocarbon-based effluents containing C2-C10 olefins derived from refinery or petrochemistry processes (FCC, vapor cracking, etc.). The charge may be a mixture of fractions comprising C3 FCC, C4 FCC, LCCS, LCCCS, Pygas, LCN, and mixtures, such that the content of linear olefins in the C5-fraction (C2-C5 hydrocarbons) relative to the total C2-C10 charge is less than or equal to 40% by weight.

The total olefin content in the C5-(C2-C5) fraction relative to the total C3-C10 charge supplied for the oligomerization may be greater than 40% by weight if the isoolefins are present in an amount of at least 0.5% by weight.

The total content of linear olefins may be greater than 40% by weight relative to the total charge of C2-C10 if the linear C6+ olefins (C6, C7, C8, C9, C10) are present in an amount of at least 0.5% by weight.

This charge may especially contain olefins, paraffins and aromatic compounds in all proportions, in conformity with the rules described above.

The process according to the invention may be performed without prior separation of the heaviest hydrocarbons of the charge.

The hydrocarbon-based charge will preferably contain a small amount of dienes and of acetylenic hydrocarbons, especially less than 100 ppm of diene, preferably less than 10 ppm of C3-C5 dienes.

To this end, the hydrocarbon-based charge will be treated, for example, by selective hydrogenation optionally combined with adsorption techniques.

The hydrocarbon-based charge will preferably contain a small amount of metals, for example less than 50 ppm and preferably less than 10 ppm.

To this end, the hydrocarbon-based charge will be treated, for example, by selective hydrogenation optionally combined with adsorption techniques.

Advantageously, the charge used has undergone a partial extraction of the isoolefins it contains, for example by treatment in an etherification unit, thus allowing concentration as linear olefins.

In general, commercially available olefinic charges bring about deactivation of the oligomerization catalyst that is faster than expected. Although the reasons for such a deactivation are not clearly understood, it is considered that the presence of certain sulfur compounds is at least partly responsible for this drop in activity and selectivity. In particular, it would appear that aliphatic thiols, sulfides and disulfides of low molecular weight are more particularly troublesome.

It is thus established that the acceptable sulfur content in a charge of an oligomerization process must be low enough for the activity of the catalyst used not to be inhibited. In general, the sulfur content is less than or equal to 100 ppm, preferably less than or equal to 10 ppm, or even less than or equal to 1 ppm.

The removal of these sulfur compounds requires hydrotreatment steps that increase the total cost of the process, and which may lead to a reduction in the amount of olefins. This loss may prove to be very penalizing for C5-C10 fractions typically containing from 200 to 400 ppm of sulfur.

There is thus also a need to develop an oligomerization process that allows the treatment of commercially available olefinic charges without a severe prior hydrotreatment.

It is common practice to add water to the charge of a catalytic oligomerization process. This addition of water makes it possible especially to control the temperature of the oligomerization reactor, in particular during the startup of the reactor, when the catalyst is fresh and the exothermicity is greatest. The presence of water-precursor oxygenated compounds in the charge used for the process according to the invention has the advantage of increasing the sulfur tolerance of the oligomerization catalysts. The lifetime of the catalyst may thus be increased. As a result of the contents of oxygenated compounds used, the water formed during the oligomerization represents more than 0.25% by weight of the hydrocarbon-based charge.

By way of example, in order to prevent the catalytic activity of the catalyst from being substantially inhibited, the nitrogen content of the hydrocarbon-based charge is not greater than 1 ppm by weight (calculated on an atomic basis), preferably not greater than 0.5 ppm and more preferably 0.3 ppm. Furthermore, by way of example, the chloride content of the hydrocarbon-based charge is not greater than 0.5 ppm by weight (calculated on an atomic basis), preferably not greater than 0.4 ppm and more preferably 0.1 ppm.

To this end, the hydrocarbon-based charge used may have undergone a prior treatment, for example a partial hydrotreatment, a selective hydrogenation and/or a selective adsorption.

The effluents of the oligomerization process will then be conveyed to a separation zone, in order to separate, for example, the fractions into an aqueous fraction, C5-C9 (gasoline), C10-C12 (jet fuel) and C12+ (diesel). The fractions C5-C9, C10-C12 and C12+ may undergo drying.

Thus, the invention makes it possible especially to obtain a jet fuel (C10-C12) from alcohols of plant origin.

The fractions C10-C12 and C12+ separated from the effluents of the oligomerization process may undergo a hydrogenation in order to saturate the olefinic compounds and to hydrogenate the aromatic compounds. The product obtained has a high cetane number and excellent properties for use as a fuel of jet or diesel type, or the like.

In one embodiment, the effluents derived from the step of oligomerization of the charge are conveyed into a separation zone in which at least the C2-C4 and/or C5-C9 olefins are separated out, and in which at least part of the C2-C4 and/or C5-C9 olefins is recycled as charge for the step of oligomerization of the hydrocarbon-based charge, optionally after having undergone an oligomerization.

In another embodiment, the hydrocarbon-based charge is oligomerized in two oligomerization reactors in series, the effluent leaving the second reactor being conveyed into a separation zone in which at least the C2-C4 and C5-C9 olefins are separated out, and in which at least part of the C2-C4 or C5-C9 olefins is recycled as charge for the first reactor of the step for oligomerization of the hydrocarbon-based charge, and at least part of the C5-C9 or C2-C4 olefins is recycled as charge for the second reactor of the step for oligomerization of the hydrocarbon-based charge.

In one variant comprising two oligomerization reactors, it may be envisioned to separate out only the C2-C4 or C5-C9 olefins, and optionally to recycle the separated olefins as charge for the first or second reactor.

The oligomerization of a charge rich in C2-C10 olefins in the presence of oxygenated compounds according to the process of the invention makes it possible to improve the management of the exothermicity of the oligomerization and thus to optimize the conversions of each reaction zone and to reduce the total energy expenditure, and similarly the corresponding investment costs. It turns out, in effect, that the reactions for conversion of the oxygenated compounds into hydrocarbon that take place are endothermic, whereas the oligomerization reactions are exothermic: the latter reactions then supply the energy required for the reactions for conversion of the oxygenated compounds.

For example, when the oxygenated compounds are alcohols, these alcohols undergo endothermic dehydration reactions leading to the formation of heavier alkenes and water.

In particular, the dehydration of ethanol (highly endothermic) leads to the formation of ethylene and water. Whereas ethylene is usually sparingly converted in the oligomerization processes, it appears that its degree of conversion may be improved in the presence of olefins, under suitable reaction conditions, especially at high temperature and low pressure. Moreover, if the oligomerization of charges containing only ethylene leads to the formation of distillates formed from highly branched hydrocarbons, the presence of heavy olefins makes it possible to reduce the degree of branching of the hydrocarbons obtained and to improve the quality of the distillate.

The process according to the invention may thus make it possible to incorporate ethylene into a hydrocarbon-based charge via an alkylation. Furthermore, the presence of aromatic compounds in the charge makes it possible to increase the degree of incorporation of ethylene into the distillates.

Moreover, the presence of oxygenated compounds in the hydrocarbon-based charge of the oligomerization process increases the partial pressure of olefins, which makes it possible to improve the yield for the oligomerization process.

The process according to the invention may be performed under the conditions described below.

Advantageously, the hydrocarbon-based charge is oligomerized by being placed in contact with an acidic catalyst in the presence of a reducing compound, such as hydrogen. In particular, the presence of a reducing atmosphere, and possibly of water, improves the stability of the catalyst used.

The mass throughput through the oligomerization reactor(s) will advantageously be sufficient to enable a relatively high conversion, without being too low, so as to avoid adverse side reactions. The hourly space velocity (weight hourly space velocity, WHSV) of the charge will be, for example, from 0.1 to 20 h⁻¹, preferably from 0.5 to 10 h⁻¹ and more preferably from 1 to 8 h⁻¹.

The temperature at the reactor inlet will advantageously be sufficient to allow a relatively high conversion, without being very high, so as to avoid adverse side reactions. The temperature at the reactor inlet will be, for example, from 150° C. to 400° C., preferably 200-350° C. and more preferably from 220 to 350° C.

The pressure across the oligomerization reactor(s) will advantageously be sufficient to allow a relatively high conversion, without being too low, so as to avoid adverse side reactions. The pressure across the reactor will be, for example, from 8 to 500 bara (0.8 to 50 MPa), preferably 10-150 bara (1 to 15 MPa) and more preferably from 14 to 49 bara (bar, absolute pressure) (1.4 to 4.9 MPa).

The process according to the invention has the advantage of being able to be performed in an existing installation.

For example, an installation containing several reactors may be used, in which the exothermicity of the reaction may be controlled so as to avoid excessive temperatures. Preferably, the maximum temperature difference within the same reactor will not exceed 75° C.

The reactor(s) may be of the isothermal or adiabatic type with a fixed or moving bed. The oligomerization reaction may be performed continuously in one configuration comprising a series of fixed-bed reactors mounted in parallel, in which, when one or more reactors are in service, the other reactors undergo regeneration of the catalyst.

The process may be performed in one or more reactors.

Preferably, the process will be performed using two separate reactors.

The reaction conditions for the first reactor will be chosen so as to convert part of the olefinic compounds with a low carbon number (C2-C5) into intermediate olefins (C6+).

For example, when the oxygenated compound is ethanol, the reaction conditions in this first reactor promote the conversion of ethylene into heavier olefins.

Advantageously, the first reactor will comprise a first catalytic zone and will function at high temperature, for example greater than or equal to 250° C., and at moderate pressure, for example less than 50 bar.

The second reactor will preferably operate at temperatures and pressures chosen so as to promote the oligomerization of heavy olefins to distillate. The effluent from the first reactor, comprising the unreacted olefins, the intermediate olefins, water and possibly other compounds such as paraffins and possibly a reducing gas, then undergoes an oligomerization in this second reactor comprising a second catalytic zone, which makes it possible to obtain an effluent of heavier hydrocarbons, rich in distillate.

Advantageously, the first reactor will function at a lower pressure and at a higher temperature and hourly space velocity than the second reactor.

It may optionally be envisioned to use the pressure difference between the two reactors in order to perform a flash separation step. Thus, for example, in the case where the oxygenated compound is ethanol, the unreacted ethylene and the other light gases may be readily separated out and removed from the heavier hydrocarbons forming the liquid phase. An excess of water may then optionally be removed.

For example, when the hydrocarbon-based charge is oligomerized in an installation comprising several reactors in series, the presence of part of the effluent derived from the condensation is not obligatory at the inlet of the first reactor. The condensation effluent may be injected into the medium of the first reactor and/or into the inlet of the second reactor, for example. It is important that the total amount of oxygenated compound added be greater than or equal to 0.5% by weight relative to the hydrocarbon-based charge.

All of the oxygenated compound may thus be added to the charge before it enters the oligomerization reactor(s), or partly before it enters the oligomerization reactor(s), the remaining part being added to the oligomerization reactor(s), for example as a quench.

The catalysts for the first and second reactors may be identical or different.

If the hydrocarbon-based charge is oligomerized in an installation comprising several reactors in series, the reactors of the series may be charged with the same catalyst or a different one.

As regards the nature of the catalyst, a first family of catalysts comprises an acidic catalyst either of amorphous or crystalline aluminosilicate type, or a silicoaluminophosphate, in H+ form, chosen from the following list and optionally containing alkali metals or alkaline-earth metals:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), ZSM-48, MFS (ZSM-57), MTW, MAZ, SAPO-11, SAPO-5, FAU, LTL, BETA MOR, SAPO-40, SAPO-37, SAPO-41 and the family of microporous materials composed of silica, aluminum, oxygen and possibly boron.

Zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or rare-earth metals is from 0.05% to 10% by weight and preferentially from 0.2% to 5% by weight. Preferentially, the metals used are Mg, Ca, Ba, Sr, La and Ce, which are used alone or as a mixture.

A second family of catalysts used comprises phosphate-modified zeolites optionally containing an alkali metal or a rare-earth metal. In this case, the zeolite may be chosen from the following list:

MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, FAU, LTL, BETA MOR.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, which are used alone or as a mixture.

A third family of catalysts used comprises difunctional catalysts, comprising:
a support, from the following list: MFI (ZSM-5, silicalite-1, boralite C, TS-1), MEL (ZSM-11, silicalite-2, boralite D, TS-2, SSZ-46), ASA (amorphous silica-alumina), MSA (mesoporous silica-alumina), FER (Ferrierite, FU-9, ZSM-35), MTT (ZSM-23), MWW (MCM-22, PSH-3, ITQ-1, MCM-49), TON (ZSM-22, Theta-1, NU-10), EUO (ZSM-50, EU-1), MFS (ZSM-57), ZSM-48, MTW, MAZ, BETA, FAU, LTL, MOR, and microporous materials of the family ZSM-48 consisting of silicon, aluminum, oxygen and optionally boron. MFI or MEL (Si/Al>25), MCM-41, MCM-48, SBA-15, SBA-16, SiO2, Al2O3, hydrotalcite, or a mixture thereof;
a metallic phase (Me) to a proportion of 0.1% by weight, the metal being selected from the following elements: Zn, Mn, Co, Ni, Ga, Fe, Ti, Zr, Ge, Sn and Cr used alone or as a mixture. These metal atoms may be inserted into the tetrahedral structure of the support via the tetrahedral unit [MeO₂]. The incorporation of this metal may be performed either by adding this metal during the synthesis of the support, or it may be incorporated after synthesis by ion exchange or impregnation, the metals then being incorporated in the form of cations, and not integrated into the structure of the support.

The zeolite may be subjected to various treatments before use, which may be: ion exchange, modification with metals, steam treatment (steaming), acid treatments or any other dealumination method, surface passivation by deposition of silica, or any combination of the abovementioned treatments.

The content of alkali metals or of rare-earth metals is from 0.05% to 10% by weight and preferentially from 0.2% to 5% by weight. Preferentially, the metals used are Mg, Ca, Ba, Sr, La and Ce, used alone or as a mixture.

The catalyst may be a mixture of the materials described previously in the three families of catalyst. In addition, the active phases may themselves also be combined with other constituents (binder, matrix) giving the final catalyst increased mechanical strength, or improved activity.

The oligomerization process according to the invention is thus a process of oligomerization under heterogeneous catalysis.

The invention is now described with reference to the examples and the attached drawings, which are not limiting, in which:

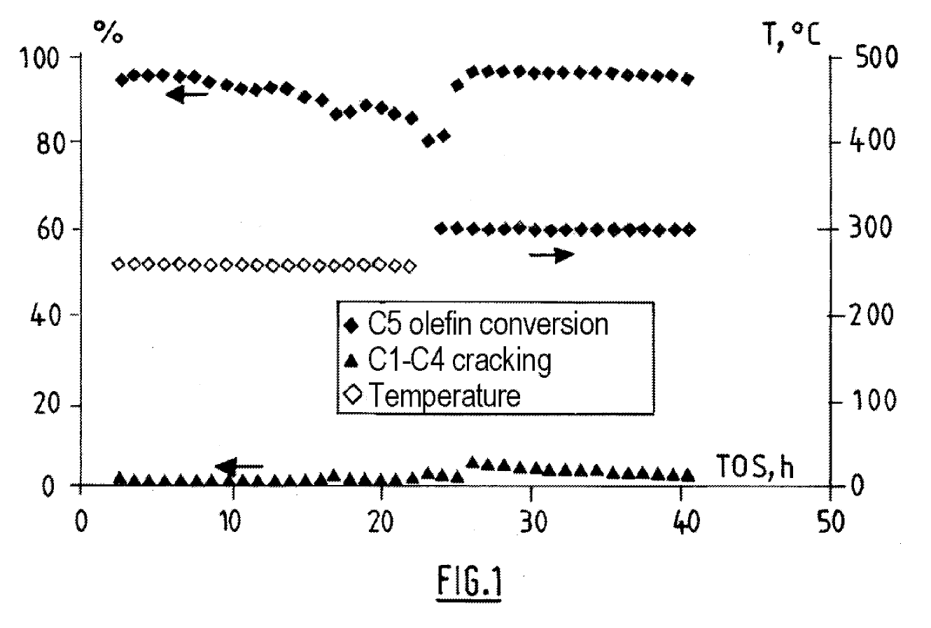

FIG. 1 represents the degree of conversion of C5 olefins at 300° C. starting from LCCS, and also the amount of cracked products (C1-C4) for Example 3, as a function of the test time (TOS);

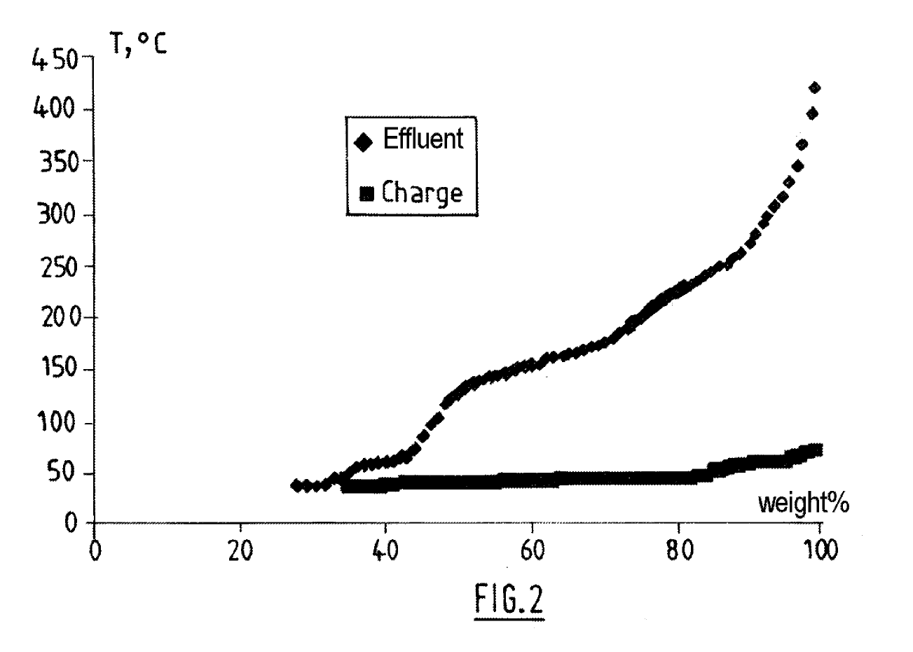

FIG. 2 represents the curve of simulated distillation of the liquid organic phase of the effluent and of the charge for Example 3;

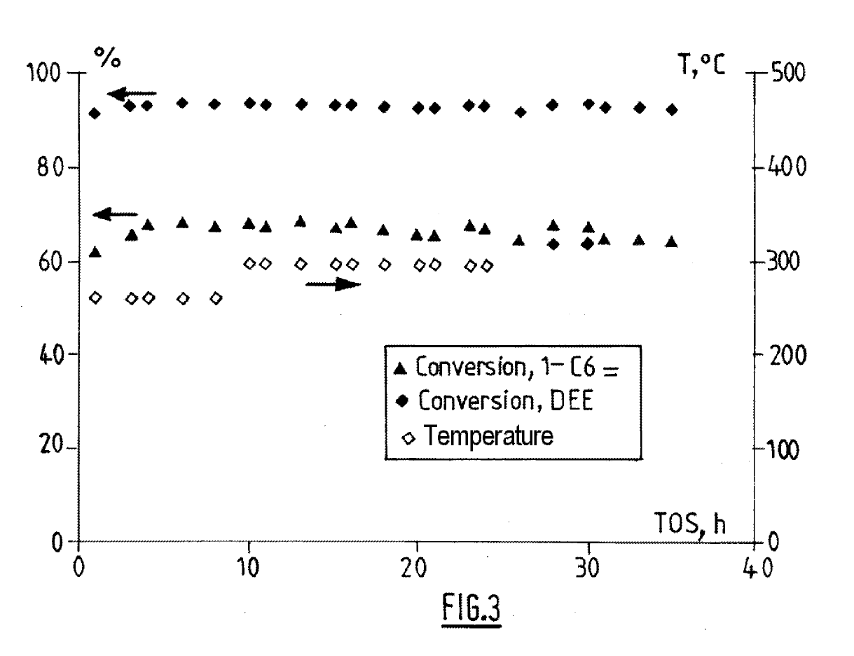

FIG. 3 represents the degrees of conversion into 1-hexene and into DEE for Example 4 as a function of the test time (TOS);

FIG. 4 represents the curve of simulated distillation for the liquid organic phase of the effluent and the charge for Example 4;

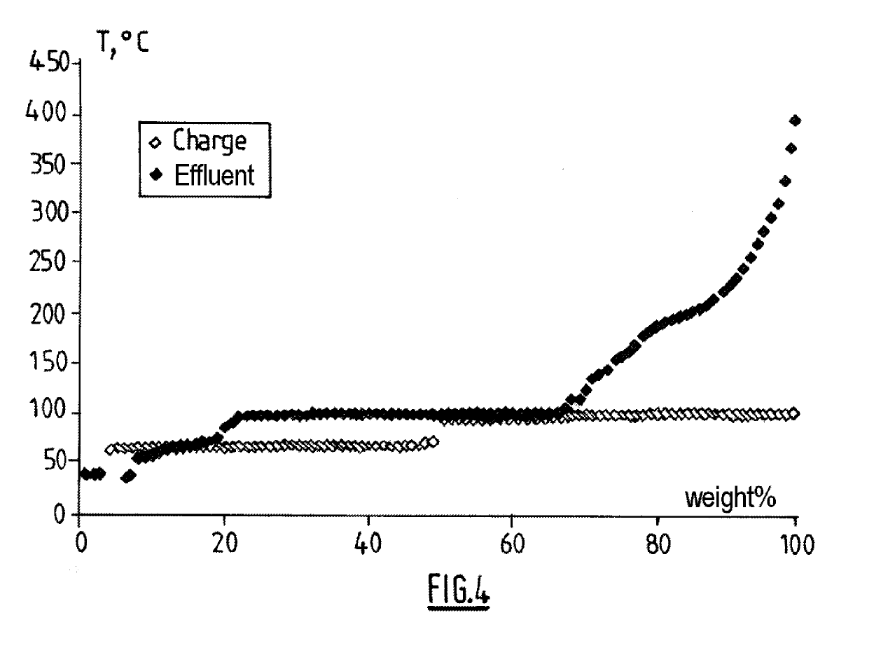

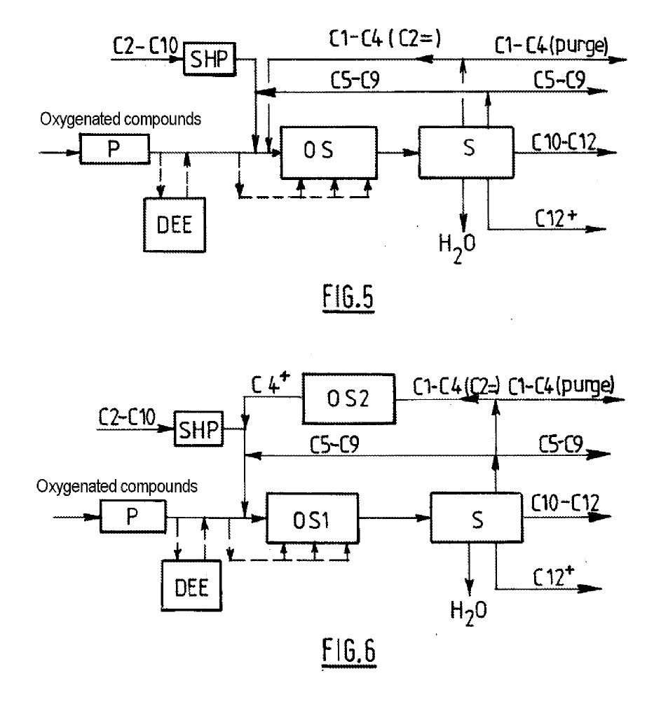

FIGS. 5 to 8 schematically represent different embodiments of the process according to the invention.

In each of the FIGS. 5 to 8:
OS represents an oligomerization zone, FIGS. 6 to 8 comprising two oligomerization zones, OS1 et OS2,
S represents a separation zone,
SHP represents a zone of selective hydrogenation and/or of selective adsorption,
DEE is a reactor for the production of diethyl ether from the oxygenated compounds,
P represents a zone for purification of the oxygenated compounds.

On these figures, the dashed lines represent process options.

Each oligomerization zone represents, for example, an oligomerization reactor.

The scheme represented in FIG. 5 corresponds to a process in which the charge consisting of C2-C10 hydrocarbon-based compounds is mixed, after selective hydrogenation (SHP), with oxygenated compounds, optionally comprising DEE, and then treated in an oligomerization zone OS. The effluent leaving this zone OS is conveyed into the separation zone S.

In this zone S, the water is removed and the olefins are separated into C2-C4 (containing essentially C2=), C5-C9 (gasoline), C10-C12 (jet) and diesel (C12+). Part of the light C2-C4 olefins, and optionally part of the C5-C9 olefins, are recycled as charge for the oligomerization zone OS.

All the oxygenated compounds (optionally comprising DEE) may be added at the inlet of the zone OS or inside this zone (dashed lines).

The process represented schematically in FIG. 6 comprises two oligomerization zones OS1 and OS2.

The charge consisting of C2-C10 hydrocarbon-based compounds is mixed, after selective hydrogenation (SHP), with oxygenated compounds, optionally comprising DEE, and then treated in a first oligomerization zone OS1. The effluent leaving this zone OS1 is conveyed into the separation zone S.

In this zone S, the water is removed and the olefins are separated into C2-C4 (containing essentially C2=), C5-C9 (gasoline), C10-C12 (jet) and diesel (C12+). Part of the light C2-C4 olefins thus separated out is sent as charge for the second oligomerization zone OS2 whose effluent, rich in C4+ olefins, is conveyed as charge for the first oligomerization zone OS1.

All the oxygenated compounds (optionally comprising the DEE) may be added at the inlet of zone OS1 or inside this zone (dashed lines)

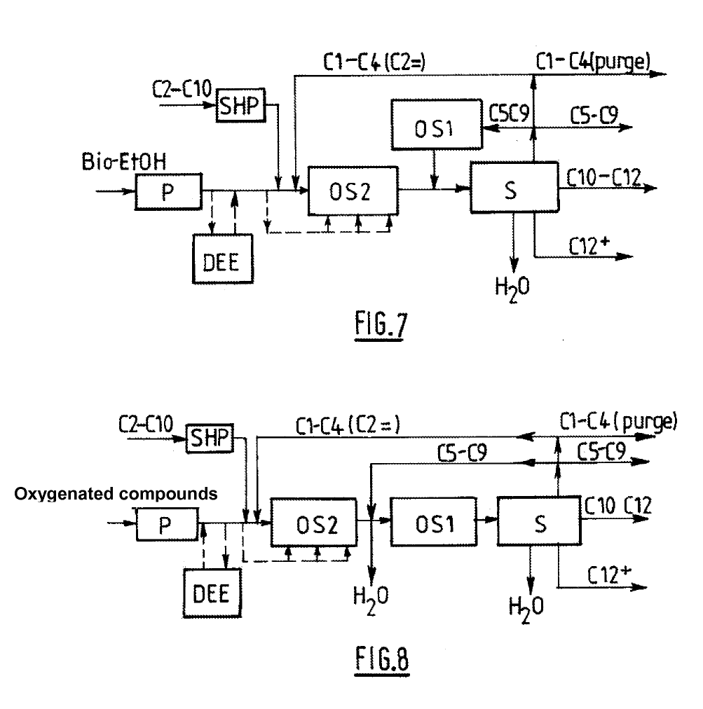

The process represented schematically in FIG. 7 comprises two oligomerization zones OS1 and OS2.

The charge consisting of C2-C10 hydrocarbon-based compounds is mixed, after selective hydrogenation (SHP), with oxygenated compounds (for example bioethanol), optionally comprising DEE, and then treated in an oligomerization zone OS2. The effluent leaving this zone OS2 is conveyed into the separation zone S.

In this zone S, the water is removed and the olefins are separated into C2-C4 (containing essentially C2=), C5-C9 (gasoline), C10-C12 (jet) and diesel (C12+).

Part of the light C2-C4 olefins thus separated out is sent as charge for the oligomerization zone OS2, whereas part of the C5-C9 olefins is sent as charge for the other oligomerization zone OS1 whose effluent is conveyed as charge for the separation zone S.

All the oxygenated compounds (optionally comprising the DEE) may be added at the inlet of the zone OS2 or inside this zone (dashed lines)

The process represented schematically in FIG. 8 comprises two oligomerization zones OS1 and OS2.

The charge consisting of C2-C10 hydrocarbon-based compounds is mixed, after selective hydrogenation (SHP), with oxygenated compounds, optionally comprising DEE, and then treated in an oligomerization zone OS2. The effluent leaving this zone OS2 is conveyed as charge for the other oligomerization zone OS1. The effluent leaving the zone OS 1 is conveyed into the separation zone S.

In this zone S, the water is removed and the olefins are separated into C2-C4 (containing essentially C2=), C5-C9 (gasoline), C2-C12 (jet) and diesel (C12+).

Part of the light C2-C4 olefins thus separated out is sent as charge for the oligomerization zone OS2, whereas part of the C5-C9 olefins is sent as charge for the oligomerization zone OS1.

In one variant, not shown, part of the light C2-C4 olefins (containing essentially ethylene) is separated out and sent as charge for the oligomerization zone OS1, whereas part of the C5-C9 olefins is sent as charge for the oligomerization zone OS2.

All the oxygenated compounds (optionally comprising DEE) may be added at the inlet of zone OS2 or inside this zone (dashed lines).

The various embodiments described above may be combined, especially their recycles.

### EXAMPLES

### Example 1

### Preparation of Catalyst A

A sample of zeolite MFI (Si/Al=82) with a crystal size of 0.2-0.3 μm supplied by Zeolyst Int. in the form of NH₄ was calcined at 550° C. for 6 hours in order to convert it into H form. The product thus obtained is named catalyst A.

### Example 2

### Preparation of Catalyst B

A sample of zeolite MFI (Si/Al=82) with a crystal size of 0.2-0.3 μm supplied by Zeolyst Int. in the form of NH₄ was exchanged with an aqueous nickel acetate solution with stirring for 4 hours at 80° C. (1 g zeolite-5 ml H₂O-0.1 g NiAc₂, 4 h, 80° C.). The catalyst was separated from the solution by filtration, dried at 110° C. for 16 hours and calcined for 6 hours at 550° C. The product thus obtained is named catalyst B.

### Example 3

### Oligomerization Test in the Presence of Butanol

20 ml (12.8 g) of catalyst A in the form of grains (35-45 mesh) were placed in a fixed-bed tubular reactor with an inside diameter of 11 mm. Before the tests, the catalyst was activated at 550° C. under a stream of nitrogen for 6 hours. After activation, the reactor was cooled to 40° C. The catalyst was placed in contact with the charge at 40° C. and at atmospheric pressure for 1 hour. Next, the pressure was increased up to the reaction value and the reactor was heated to 200° C. at a rate of 30° C./hour. The temperature was maintained for 12 hours at 200° C. and was then increased up to 260° C. (30° C./hour).

The charge used for this oligomerization test is a fraction LLCCS containing 83% by weight of C5 hydrocarbons (of which 59% by weight are olefins and 41% by weight are paraffins). The content of linear olefins in the C5 fractions is 27.2% by weight.

The mixture comprising 85% by weight of LLCCS and 15% by weight of 1-butanol was placed in contact with catalyst A under the following conditions:

| | |
|---|---|
| Reactor inlet temperature: | 260, 300° C. |
| Pressure P: | 40 barg |
| Hourly space velocity (pph): | 1 h⁻¹. |

(P(barg)=P bar−Patm(˜1 bar))

The analysis of the products obtained was performed online by gas chromatography, the chromatograph being equipped with a capillary column.

At the reactor outlet, the gaseous phase, the liquid organic phase and the aqueous phase were separated. No recycling was performed.

The catalyst showed very little deactivation, which may be compensated for by increasing the temperature without substantially increasing the gaseous phase (FIG. 1).

The curve of simulated distillation of the liquid organic phase is reported in FIG. 2.

Table 1 below collates the degrees of conversion and the yields obtained.

TABLE 1

| Conversion of 1-butanol into | 99.9 | |
|---|---|---|
| HC (weight %) | | |
| Conversion of C5 (weight %) | >95% | |
| | Relative to | |
| Yields (weight %) | carbon | Relative to the olefins |
| C1-C3 | 0.1 | 0.2 |
| nC4 | 0.5 | 0.8 |
| Total C4 | 1.2 | 1.9 |
| Gasoline (distillate <150° C.) | 57.3 | 32.4 |
| Diesel (distillate >150° C.) | 41.5 | 65.6 |

This Table 1 illustrates the possibility of producing a distillate-rich heavy hydrocarbon fraction from a charge containing a gasoline fraction and 15% butanol. The charge contained several olefins with different reactivities. The results show a virtually total conversion of butanol into hydrocarbons, little cracking (gaseous phase) despite a relatively high temperature for the oligomerization, and the conversion of a significant amount (65.6%) of olefins distilling above 150° C. from a real charge.

Table 2 below collates the compositions of the gasoline fractions before and after oligomerization. These data show that the gasoline produced after oligomerization has a research octane number (RON) and a motor octane number (MON) close to those of the gasoline before oligomerization, contains a smaller amount of olefins and has a very low benzene content.

TABLE 2

| | | Gasoline fraction obtained |
|---|---|---|
| | LLCCS | after oligomerization |
| | (Weight %) | (Weight %) |
| Isoparaffins | 34.2 | 55.3 |
| Normal paraffins | 5.4 | 7.3 |
| Cyclic paraffins | 0.7 | 4.1 |
| Isoolefins | 29.2 | 20.6 |
| Normal olefins | 28.0 | 1.3 |
| Cyclic olefins | 2.4 | 1.3 |
| Total aromatics | 0.04 | 10.5 |
| Benzene | 0.042 | 0.034 |
| Paraffins | 40.3 | 62.6 |
| Olefins | 59.6 | 22.0 |
| Aromatics | 0.04 | 10.5 |

### Example 4

### Oligomerization Test in the Presence of Diethyl Ether

10 ml (6.3 g) of catalyst B in the form of grains (35-45 mesh) were placed in a fixed-bed tubular reactor with an inside diameter of 11 mm. Before the tests, the catalyst was activated at 550° C. under a stream of nitrogen for 6 hours. After activation, the reactor was cooled to 40° C. The catalyst was placed in contact with the charge at 40° C. and at atmospheric pressure for 1 hour. Next, the pressure was increased to the reaction value and the reactor was heated up to 200° C. at a rate of 30° C./hour. The temperature was maintained for 12 hours at 200° C. and was then increased up to 260, 300 and 320° C. (30° C./hour).

The charge used for this oligomerization test is a mixture of 50% by weight of 1-hexene and 50% by weight of n-heptane. The content of linear olefins in the C5-fractions in the charge was 0% by weight.

The oxygenated compound tested is DEE (diethyl ether).

85% by weight of the synthetic mixture (1-hexene/n-heptane) and 15% by weight of DEE were placed in contact with catalyst A under the conditions collated in Table 3.

The analysis of the products obtained was performed online by gas chromatography, the chromatograph being equipped with a capillary column.

At the reactor outlet, the gaseous phase, the liquid organic phase and the aqueous phase were separated. No recycling was performed. The catalyst showed very little deactivation, which may be compensated for by increasing the temperature without substantially increasing the gaseous phase (FIG. 3).

FIG. 4 represents the curve of simulated distillation of all of the organic phase obtained from the charge at a reaction temperature of 300° C. The curves were similar for the various reactor inlet temperatures.

Table 3 below collates the degrees of conversion and the selectivities obtained.

This table illustrates the possibility of producing heavy hydrocarbons from a charge containing a synthetic charge mixed with 15% by weight of oxygenated compounds such as DEE or ethanol. The results show that about 66% by weight of carbon from n-hexene are incorporated into the liquid effluent.

A possibility of working at high temperature (up to 320° C.) without causing a substantial increase in the gaseous phase and a drop in selectivity toward oligomers was demonstrated in FIG. 3 and in Table 3.

TABLE 3
Conditions

| Charge, weight % | 15.0 DEE + 42.5 1-C6═ + 42.5 C7 | | |
|---|---|---|---|
| Charge, weight % of | 11.8 DEE + 44.1 1-C6═ + 44.1 C7 | | |
| carbon | | | |
| PPH, h⁻¹ | 1 | 1 | 1 |
| P, barg | 40 | 40 | 40 |
| Tᵢₙₗₑₜ, ° C. | 260 | 300 | 320 |

Conversion, % (weight)

| | | | |
|---|---|---|---|
| 1-C6═ | 67.4 | 67.2 | 65.6 |
| DEE | 93.2 | 93.1 | 92.8 |

Selectivity, % (weight)

| | | | |
|---|---|---|---|
| Contribution of DEE | 27.1 | 27.0 | 27.4 |
| Contribution of 1-C6═ | 72.9 | 73.0 | 72.6 |
| C1-C4 | 9.2 | 9.3 | 9.5 |
| C7-C11 | 24.4 | 26.3 | 25.0 |
| Group C12 | 34.5 | 37.5 | 37.2 |
| Group C18 | 14.4 | 13.9 | 13.8 |
| Group C24 | 14.5 | 13.0 | 14.6 |
| Group C30 | 0.0 | 0.0 | 0.0 |

1. A process for producing distillates from a hydrocarbon-based charge containing C2-C10 olefins, in which the treatment of the charge comprises at least one step of oligomerization of the charge performed in at least one oligomerization reactor, in which the charge is oligomerized in the presence of at least 0.5% by weight of at least one oxygenated organic compound containing at least one oxygen atom and at least two carbon atoms, this oxygenated organic compound being derived from a synthetic step performed before the oligomerization step, the organic compound possibly being of plant origin.

2. The process as claimed in claim 1, in which the hydrocarbon-based charge is oligomerized in the presence of not more than 70% by weight of oxygenated compound(s), preferably from 0.5% to 50% by weight and more particularly from 1% to 30% by weight.

3. The process as claimed in claim 1, in which the organic compound is chosen from alcohols, ethers, with the exception of dimethyl ether, carbonyl compounds, especially of C2-C20 and preferably C2-C8, and the corresponding ethers.

4. The process as claimed in claim 3, in which the organic compound is chosen from alcohols or ethers, preferably from ethanol, propanol, isopropanol, butanol, isobutanol, glycerol, ethylene glycol and the corresponding ethers.

5. The process as claimed in claim 1, in which all the products obtained during the step of synthesis of the oxygenated compound(s) are mixed with the charge for the oligomerization step.

6. The process as claimed in claim 1, in which the oxygenated compound(s) are obtained by conversion of biomass.

7. The process as claimed in claim 1, in which the oxygenated compound(s) are obtained by condensation of light oxygenated molecules, possibly originating from biomass.

8. The process as claimed in claim 7, in which the weight ratio of the effluent derived from the condensation of ethanol and of the charge containing C2-C10 olefins is from 0.005 to 1000 and preferably from 0.01 to 100.

9. The process as claimed in claim 1, in which the hourly space velocity of the charge is between 0.1 and 20 h⁻¹, preferably from 0.5 to 15 h⁻¹ and more preferably from 1 to 8 h⁻¹.

10. The process as claimed in claim 1, in which the reactor inlet temperature is from 150 to 400° C., preferably 200-350° C. and more preferably from 220 to 350° C.

11. The process as claimed in claim 1, in which the pressure in the reactor(s) is from 8 to 500 bara, preferably 10-150 bara and more preferably from 14 to 49 bara.

12. The process as claimed in claim 1, in which the hydrocarbon-based charge is oligomerized by means of two reactors, the reaction conditions of the first reactor being chosen so as to convert part of the olefinic compounds with a low C2-C5 carbon number into intermediate C6+ olefins.

13. The process as claimed in claim 12, in which the second reactor functions at temperatures and pressures chosen so as to promote the oligomerization of the heavy olefins into distillate.

14. The process as claimed in claim 12, in which the first reactor functions at a lower pressure and a higher temperature and hourly space velocity relative to the second reactor.

15. The process as claimed in claim 12, in which the pressure difference between the two reactors is sufficient to make it possible to perform a flash separation of the effluent leaving the first reactor in order to remove therefrom the light gases, and optionally an excess of water, before introducing it into the second reactor.

16. The process as claimed in claim 1, in which the effluents derived from the step for oligomerization of the charge are conveyed into a separation zone in which at least the C2-C4 and/or C5-C9 olefins are separated out, and in which at least part of the C2-C4 and/or C5-C9 olefins is recycled as charge for the step of oligomerization of the hydrocarbon-based charge, optionally after having undergone an oligomerization.

17. The process as claimed in claim 1, in which the hydrocarbon-based charge is oligomerized in two oligomerization reactors in series, the effluent leaving the second reactor being conveyed into a separation zone in which at least the C2-C4 and C5-C9 olefins are separated out, and in which at least part of the C2-C4 or C5-C9 olefins is recycled as charge for the first reactor of the step for oligomerization of the hydrocarbon-based charge, and at least part of the C5-C9 or C2-C4 olefins is recycled as charge for the second reactor of the step for oligomerization of the hydrocarbon-based charge.
